(19) United States
(12) Patent Application Publication
(10) Pub. No.: US 2023/0001081 A1
(43) Pub. Date: Jan. 5, 2023

(54) PUMPS WITH ORIENTATION INDEPENDENT LIQUID DRUG ACCESSIBILITY

(71) Applicant: JANSSEN BIOTECH, INC., Horsham, PA (US)

(72) Inventors: Jimmy NGUYEN, Fremont, CA (US); Brandon MCKENZIE, West Chester, PA (US)

(21) Appl. No.: 17/784,418

(22) PCT Filed: Nov. 23, 2020
(86) PCT No.: PCT/IB2020/061017

§ 371 Date: Jun. 10, 2022

Related U.S. Application Data
(60) Provisional application No. 62/950,301, filed on Dec. 19, 2019.

(51) Int. Cl. A61M 5/142; A61M 39/08

(52) U.S. Cl. CPC A61M 5/14244; A61M 39/08

(57) ABSTRACT

Various exemplary pumps with orientation independent liquid drug accessibility are provided. In general, a pump includes a reservoir configured to contain a liquid drug therein, a conduit configured to receive the drug therein from the reservoir, and a needle in fluid communication with the conduit and configured to deliver the drug therethrough to a patient wearing the pump. The conduit includes a mechanism configured to ensure that the conduit is in complete communication with the drug in the reservoir at least when the conduit is receiving the drug therein from the reservoir regardless of the patients orientation. In one exemplary embodiment, the mechanism includes a weight attached to the conduit. In another exemplary embodiment, the mechanism includes a fork at a free end of the conduit that is located in the reservoir.

### FIELD

The present disclosure relates generally to pumps with orientation independent liquid drug accessibility.

### BACKGROUND

Pharmaceutical products (including large and small molecule pharmaceuticals, hereinafter “drugs” or “therapeutic substances”) are administered to patients in a variety of different ways for the treatment of specific medical indications. A pump is a type of drug administration device that can administer a liquid drug to the patient. Some pumps are wearable by a patient and can include a reservoir, such as a vial or a cartridge, that contains the liquid drug therein for delivery to the patient through a needle inserted into the patient.

The drug can be removed from the reservoir through a conduit and delivered to the patient through the needle. However, if the conduit is not in complete communication with the liquid drug in the reservoir, air can enter the conduit with the drug or instead of the drug. Air is undesirable to deliver to the patient because of, e.g., patient discomfort. If the conduit is not in complete communication with the liquid drug in the reservoir, the patient's desired treatment is interrupted by the pump delivering only air to the patient instead of the drug, by the pump delivering air to the patient with only a partial intended dose of the drug, or by the pump not delivering any air or any drug to the patient due to a detected error of air entering the conduit from the reservoir. Interrupting the patient's treatment may adversely affect the patient's health and may cause patient frustration with the pump and thereby reduce the patient's willingness to use the pump in the future as recommended by the patient's health care provider.

The conduit may not be in complete communication with the liquid drug in the reservoir for a variety of reasons. For example, the conduit may not be in complete communication with the liquid drug in the reservoir due to the patient's orientation when the drug is being pumped out of the reservoir and into the patient via the needle. Liquid in the reservoir naturally settles at a location therein due to gravity, so depending on the patient's orientation, the liquid drug may not settle within the reservoir at a location where the conduit is in complete communication with the liquid drug. Additionally, for pumps that deliver multiple doses of the drug over time, it becomes more likely over time that the patient's orientation will adversely affect the conduit's accessibility of the drug within the reservoir. As the amount of the drug in the reservoir decreases, there is less drug present in the reservoir to be in complete communication with the conduit.

For another example, the conduit may not be in complete communication with the liquid drug in the reservoir due to the pump not being positioned properly on the patient. The pump will typically come with instructions indicating how the pump should be attached to the patient, including a recommended orientation of the pump relative to the patient. The recommended orientation may help maximize the conduit's ability to be in complete communication with the drug in the reservoir for every delivery of the drug to the patient. However, the pump may not be attached to the patient at the recommended orientation due to unintentional user error.

Accordingly, there remains a need for pumps with improved liquid drug accessibility.

### SUMMARY

In general, pumps with orientation independent liquid drug accessibility are provided.

In one aspect, a pump configured to deliver a drug to a patient is provided that in one embodiment includes a reservoir configured to contain a liquid drug therein, and a conduit configured to receive the drug therein from the reservoir. The conduit has a weight at a free end thereof. The pump also includes a needle configured to be inserted into a patient, and a pumping assembly configured to drive the liquid drug into the conduit from the reservoir and into the needle for delivery of the liquid drug into the patient. The pump can have any number of variations.

In another embodiment, a pump configured to deliver a drug to a patient includes a reservoir configured to contain a liquid drug therein, and a conduit configured to receive the drug therein from the reservoir. The conduit includes a single proximal passageway therein. A free end of the conduit includes a plurality of tubular prongs each distal to the single proximal passageway and each including a secondary passageway therein. Each of the secondary passageways is in fluid communication with the single proximal passageway. The pump also includes a needle configured to be inserted into a patient, and a pumping assembly configured to drive the liquid drug into the conduit from the reservoir and into the needle for delivery of the liquid drug into the patient. The pump can have any number of variations.

In yet another embodiment, a pump configured to deliver a drug to a patient includes a reservoir configured to contain a liquid drug therein, a conduit configured to receive the drug therein from the reservoir, a telescoping cannula configured to slide in and out of the conduit in response to gravitational force, a needle configured to be inserted into a patient, and a pumping assembly configured to drive the liquid drug into the conduit from the reservoir and into the needle for delivery of the liquid drug into the patient. The pump can have any number of variations.

In another aspect, a method of using a pump configured to deliver a drug to a patient is provided and in one embodiment includes activating a pumping assembly of the pump to move a liquid drug into a conduit of the pump from a reservoir and from the conduit into a needle of the pump. The method can have any number of variations.

### BRIEF DESCRIPTION OF DRAWINGS

The present invention is described by way of reference to the accompanying figures which are as follows:

FIG. 1 is a schematic view of an embodiment of a pump configured to deliver a liquid drug to a patient;

FIG. 2A is a schematic view of an embodiment of a reservoir of the pump of FIG. 1 in various orientations;

FIG. 2B is a perspective view of an area of accessibility for a conduit of the pump of FIG. 1;

FIG. 3 is a perspective schematic view of an embodiment of a conduit of the pump of FIG. 1;

FIG. 4 is a perspective schematic view of another embodiment of a conduit of the pump of FIG. 1;

FIG. 5 is a perspective schematic view of yet another embodiment of a conduit of the pump of FIG. 1;

FIG. 6 is a schematic view of still another embodiment of a conduit of the pump of FIG. 1,

FIG. 7 is a schematic view of another embodiment of the conduit of the pump of FIG. 1;

FIG. 8 is a schematic view of yet another embodiment of the conduit of the pump of FIG. 1, the conduit being in an initial position;

FIG. 9 is a schematic view of the conduit of FIG. 8 in an intermediate position;

FIG. 10 is a schematic view of the conduit of FIG. 9 in a delivery position;

FIG. 11 is a schematic view of yet another embodiment of the conduit of the pump of FIG. 1 attached to a telescoping cannula, the telescoping cannula being in a first position;

FIG. 12 is a schematic view of the conduit and telescoping cannula of FIG. 11 in a second position;

FIG. 13 is a schematic view of the conduit and telescoping cannula of FIG. 12 in a third position;

FIG. 14 is a schematic view of the conduit and telescoping cannula of FIG. 13 in a fourth position;

FIG. 15 is a schematic view of another embodiment of a pump configured to deliver a liquid drug to a patient and an embodiment of a reservoir configured to be received in the pump;

FIG. 16 is a schematic view of the reservoir and pump of FIG. 15 coupled together; and

FIG. 17 is a schematic view of the reservoir and pump of FIG. 16 with a conduit of the pump penetrated into the reservoir.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Various exemplary pumps with orientation independent liquid drug accessibility are provided. In general, a pump includes a reservoir configured to contain a liquid drug therein, a conduit configured to receive the drug therein from the reservoir, and a needle in fluid communication with the conduit and configured to deliver the drug therethrough to a patient wearing the pump. The conduit includes a mechanism configured to ensure that the conduit is in complete communication with the drug in the reservoir at least when the conduit is receiving the drug therein from the reservoir regardless of the pump's orientation on the patient or the patient's orientation, e.g., whether the patient is standing, sitting, lying down, bent over, etc. The mechanism is thus configured to ensure that the conduit receives therein only drug from the reservoir for delivery to the patient and that the conduit does not receive therein any air contained in the reservoir. The patient may thereby be ensured to receive only drug through the needle and not any air through the needle, and the patient's drug dose(s) can therefore be fully delivered at a desired schedule without interruption since drug, and not any air, will be provided to the needle via the conduit.

The mechanism can have a variety of configurations. In an exemplary embodiment, the mechanism includes a weight attached to the conduit. The weight can be attached to the conduit by being integrally formed with the conduit or by being a separate member fixedly attached to the conduit by being adhered thereto with adhesive, by being embedded within material forming the conduit, or by being attached to the conduit using another attachment mechanism. The weight is positioned at a free (or distal) end of the conduit that is located within the reservoir. The free end of the conduit has an opening therein into which the liquid drug enters a passageway of the conduit for delivery to the needle. The pump can be assembled with the weighted free end of the conduit located within the reservoir, or the weighted free end of the conduit can be movable after pump assembly from an initial position located outside of the reservoir to a delivery position located inside of the reservoir. In this exemplary embodiment, the conduit is formed of a flexible material that allows the conduit to flex or bend within the reservoir. The weighted conduit is therefore configured to flex or bend within the reservoir as the pump's orientation changes, with the weight facilitating the flexing or bending of the conduit by the weight being urged in a downward direction due to gravity (where “downward” indicates a direction toward the ground). Since liquid in the reservoir naturally settles at a location in the reservoir due to gravity, the weighted conduit is configured to maintain complete communication with the liquid drug regardless of the patient's orientation. In other words, the weighted conduit is configured to “follow” the liquid drug in the reservoir to the liquid drug's settled location regardless of the patient's orientation.

In another exemplary embodiment, the mechanism includes a fork at a free (or distal) end of the conduit that is located in the reservoir. The pump can be assembled with the forked free end of the conduit located within the reservoir, or the forked free end of the conduit can be movable after pump assembly from an initial position located outside of the reservoir to a delivery position located inside of the reservoir. The conduit includes a primary (or proximal) passageway in which the liquid drug flows from the conduit to the needle. The fork includes a plurality of prongs that each includes a secondary (or distal) passageway in fluid communication with the primary passageway. Each of the secondary passageways includes a distal opening into which the liquid drug enters before entering the primary passageway for delivery to the needle. The liquid drug in the reservoir can thus enter the primary passageway through any of the secondary passageways. The forked conduit thus helps ensure that at least one of the prongs is in complete communication with the liquid drug regardless of the patient's orientation and regardless of where the liquid drug has settled within the reservoir under the force of gravity.

In another exemplary embodiment, the mechanism includes a telescoping cannula attached to the conduit. The telescoping cannula includes at least one tubular member freely slidably disposed within the conduit. The one or more tubular members are each configured to freely slide in and out of a free (or distal) end of the conduit that is located within the reservoir. In embodiments with at least two tubular members, the two or more tubular members are telescoped with each other. In other words, the tubular members are nested in the conduit in telescoping fashion. A free (or distal) end of the innermost one of the tubular members (or the sole tubular member if the mechanism includes only one tubular member) has an opening therein into which the liquid drug enters for delivery from the reservoir to the needle. The pump can be assembled with the free end of the conduit and the free end of each of the one or more tubular members located within the reservoir, or the free end of the conduit and the free end of each of the one or more tubular members can be movable after pump assembly from an initial position located outside of the reservoir to a delivery position located inside of the reservoir. In this exemplary embodiment, the conduit and each of the one or more tubular members is formed of a rigid material, which may facilitate smooth sliding of the one or more tubular members in and out of the conduit. The one or more tubular members are configured to freely slide in and out of the conduit as the pump's orientation changes, with the movement of the tubular member(s) being caused by gravity. Since liquid in the reservoir naturally settles at a location in the reservoir due to gravity, the one or more tubular members are configured to maintain complete communication with the liquid drug regardless of the patient's orientation.

The drug to be delivered using a pump as described herein can be any of a variety of drugs. Examples of drugs that can be delivered using a pump as described herein include antibodies (such as monoclonal antibodies), hormones, antitoxins, substances for the control of pain, substances for the control of thrombosis, substances for the control of infection, peptides, proteins, human insulin or a human insulin analogue or derivative, polysaccharide, DNA, RNA, enzymes, oligonucleotides, antiallergics, antihistamines, anti-inflammatories, corticosteroids, disease modifying anti-rheumatic drugs, erythropoietin, and vaccines.

The mechanisms described herein can be used with a variety of drug delivery pumps configured to deliver a drug to a patient. Examples of drug delivery pumps include the pumps described in Intl. Pat. Pub. WO 2018/096534 entitled “Apparatus For Delivering A Therapeutic Substance” published May 31, 2018, in U.S. Pat. Pub. No. 2019/0134295 entitled “Local Disinfection For Prefilled Drug Delivery System” published May 9, 2019, in U.S. Pat. No. 7,976,505 entitled “Disposable Infusion Device Negative Pressure Filling Apparatus And Method” issued Jul. 12, 2011, and in U.S. Pat. No. 7,815,609 entitled “Disposable Infusion Device Positive Pressure Filling Apparatus And Method” issued Oct. 19, 2010, which are hereby incorporated by reference in their entireties. Other examples of drug delivery pumps include the SmartDose® Drug Delivery Platform available from West Pharmaceutical Services, Inc. of Exton, Pa., the OMNIPOD® available from Insulet Corp. of Acton, Mass., the YpsoDose® patch injector available from Ypsomed AG of Burgdorf, Switzerland, the BD Libertas™ wearable injector available from Becton, Dickinson and Co. of Franklin Lakes, N.J., the Sorrel Medical pump available from Sorrel Medical of Netanya, Israel, the SteadyMed PatchPump® available from SteadyMed Ltd. of Rehovot, Israel, the Sensile Medical infusion pump available from Sensile Medical AG of Olten, Switzerland, the SonceBoz wearable injectors available from SonceBoz SA of Sonceboz-Sombeval, Switzerland, enFuse® available from Enable Injections of Cincinnati, Ohio, the on-body injector for Neulasta® available from Amgen, Inc. of Thousand Oaks, Calif., the Pushtronex® System available from Amgen, Inc. of Thousand Oaks, Calif., and the Imperium® pump available from Unilife Corp. of King of Prussia, Pa.

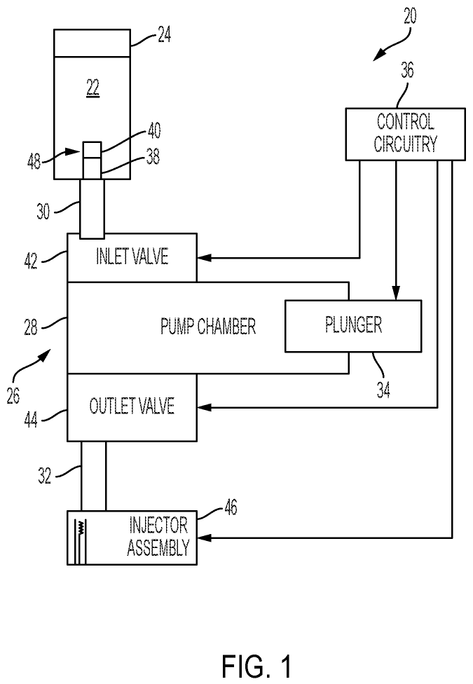

FIG. 1 illustrates an embodiment of a pump 20, e.g., a patch pump, configured to be worn by a patient and to deliver a drug (also referred to herein as a “therapeutic substance”) 22 to the patient. The pump 20 can be configured to be attached to the patient in any of a variety of ways, as will be appreciated by a person skilled in the art, such as by including a backing or label configured to be removed from a body of the pump 20 to expose adhesive attachable to the patient. The pump 20 includes a therapeutic substance reservoir 24 containing the drug 22 therein. The reservoir 24 can be prefilled by a medical vendor or device manufacturer, or the reservoir 24 can be filled by a user (e.g., the patient, the patient's caregiver, a doctor or other health care professional, a pharmacist, etc.) prior to use of the pump 20. Alternatively, the reservoir 24 can come prefilled from a medical vendor ready to be loaded or inserted into pump 20 prior to use. The pump 20 also includes a conduit 38 through which the drug 22 is configured to pass from the reservoir 24 and into an inlet fluid path 30 operatively connected to an injector assembly 46 of the pump 20 that is configured to deliver the therapeutic substance 22 into a patient. The conduit 38 is thus a tube in which the drug 22 can flow. As discussed further below, the conduit 38 includes a mechanism 40 configured to ensure that the conduit 38 is in complete communication with the drug 22 in the reservoir 24 at least when the conduit 38 is receiving the drug 22 therein from the reservoir 24, e.g., under force of an electromechanical pumping assembly 26 of the pump 20, regardless of an orientation of the patient wearing the pump 20, e.g., regardless of the pump's orientation on the patient or whether the patient is standing, sitting, lying down, bent over, etc. The mechanism 40 may therefore be configured to ensure that the drug 22, but not air, enters the conduit 38 from the reservoir 24. FIG. 1 shows the conduit 38 in complete communication with the drug 22 in the reservoir 24.

The electromechanical pumping assembly 26 is operatively connected to the reservoir 24 and is configured to cause delivery of the therapeutic substance 22 to the patient via the injector assembly 46, e.g., through a needle or cannula of the injector assembly 46 that has been inserted into the patient. The electromechanical pumping assembly 26 is shaped to define a rigid pump chamber 28 that includes a therapeutic substance inlet 30 through which the therapeutic substance 22 is received from the conduit 30, and hence from the reservoir 24, into the pump chamber 28. The rigid pump chamber 28 also includes a fluid path outlet 32 through which the therapeutic substance 22 is delivered from the pump chamber 28 to the patient via the injector assembly 46. Although the pumping assembly 26 is electromechanical in this illustrated embodiment, the pumping assembly of the pump 20 (and for other embodiments of pumps described herein) can instead be mechanical. The mechanical pumping assembly need not include any electronic components or controls. For example, the mechanical pumping assembly can include a balloon diaphragm configured to be activated to cause delivery of a drug through mechanical action.

The pump 20 also includes a plunger 34 slidably disposed within the pump chamber 28 and sealably contacting an inside of the pump chamber 28. The plunger 34 is configured to be in direct contact with the drug 22 in the pumping chamber 28.

The pump 20 also includes control circuitry 36. The electromechanical pumping assembly 26 is configured to be driven to operate in two pumping phases by the control circuitry 36. In a first pumping phase, the control circuitry 36 is configured to drive the plunger 34 (e.g., slidably move the plunger 34 in the pump chamber 28) to draw the drug 22 from the reservoir 24 into the conduit 38, then into the inlet fluid path 30, then through an inlet valve 42 and into the pump chamber 28. The inlet valve 42 is configured to be opened and closed such that when the inlet valve 42 is open there is fluid communication between the reservoir 24 and the pump chamber 28, and when the inlet valve 42 is closed there is no fluid communication between the reservoir 24 and the pump chamber 28. During the first pumping phase, the control circuitry 36 is configured to cause the inlet valve 42 to open, cause an outlet valve 44 to close, and drive the plunger 34 to draw the therapeutic substance 22 from the reservoir 24 into the pump chamber 28, e.g., the control circuitry 36 is configured to set the inlet valve 42 and the outlet valve 44 such that the therapeutic substance 22 can flow only between the reservoir 24 and the pump chamber 28. Thus, as the plunger 34 is drawn back, therapeutic substance 22 is drawn into pump chamber 28. The control circuitry 36 causing the inlet valve 42 to open and the outlet valve 44 to close can be active control or can be passive control in which the valves 42, 44 are mechanical valves that automatically open/close due to the driving of the plunger 34.

In a second pumping phase, the control circuitry 36 is configured to drive the plunger 34 to deliver the drug 22 from the pump chamber 28 through the outlet valve 44 to the outlet fluid path 32 and then to the injector assembly 46 for delivery into the patient. The outlet valve 44 is configured to be opened and closed such that when the outlet valve 44 is open there is fluid communication between the pump chamber 28 and the patient, and when the outlet valve 44 is closed there is no fluid communication between the pump chamber 28 and the patient. During the second pumping phase, the control circuitry 36 is configured to cause the inlet valve 42 to close, cause the outlet valve 44 to open, and drive the plunger 34 to deliver the therapeutic substance 22 from the pump chamber 28 in a plurality of discrete motions of the plunger 34. For example, the control circuitry 36 can be configured to set the inlet valve 42 and the outlet valve 44 such that the therapeutic substance 22 can flow only between the pump chamber 28 and the patient, and the plunger 34 is incrementally pushed back into the pump chamber 28 in a plurality of discrete motions thereby delivering the therapeutic substance 22 to the patient in a plurality of discrete dosages. Similar to that discussed above, the control circuitry 36 causing the inlet valve 42 to close and the outlet valve 44 to open can be active control or can be passive control in which the valves 42, 44 are mechanical valves that automatically open/close due to the driving of the plunger 34.

In some embodiments, the control circuitry 36 is configured to drive the plunger 34 to draw the therapeutic substance 22 into the pump chamber 28 in a single motion of the plunger 34, e.g., plunger 34 is pulled back in a single motion to draw a volume of the therapeutic substance 22 into the pump chamber 28 during the first pumping phase. Alternatively, that control circuitry 36 can be configured to drive the plunger 34 to draw the therapeutic substance 22 into the pump chamber 28 in one or more discrete expansion motions of the plunger 34, e.g., the plunger 34 can be pulled halfway out of the pump chamber 28 in one motion and then the rest of the way out of the pump chamber 28 in a second, separate motion. In this case, a duration of some or all expansion motions of the plunger 34 during the first pumping phase are typically longer than a duration of any one of the plurality of discrete motions of the plunger 34 during the second pumping phase.

In other embodiments, the control circuitry 36 is configured to drive the plunger 34 such that a duration of the first pumping phase and a duration of the second pumping phase are unequal. For example, a duration of the second pumping phase can be in a range of five to fifty times longer than the first pumping phase, e.g., at least ten times, thirty times, fifty times, etc. longer than a duration of the first pumping phase.

The pump 20 can also include a power source (not shown) configured to provide power to the control circuitry 36 and the pumping assembly 26. In an exemplary embodiment, the power source is a single power source configured to provide power to each component of the pump 20 requiring power to operate, which may help reduce cost of the pump 20 and/or conserve space within the pump 20 for other components and/or to help reduce an overall size of the pump 20. The power source can, however, include a plurality of power sources, which may help provide redundancy and/or help reduce cost of the pump 20 since some components, e.g., the control circuitry 36, may be manufactured with an on-board dedicated power supply.

The mechanism 40 can have a variety of configurations. In general, the mechanism 40 is positioned at a free (or distal) end 48 of the conduit 38 that is located within the reservoir 24. The mechanism 40 being at the free end 48 can include the mechanism 40 defining a distal-most end of the conduit 38 or the mechanism 40 being located adjacent to the distal-most end of the conduit 38. The free end 48 of the conduit 38 has an opening therein into which the drug 22 enters a passageway of the conduit 38 from the reservoir 24. The pump 20 can be assembled with the mechanism 40 located within the reservoir 24, or the mechanism 40 can be movable after pump 20 assembly from an initial position located outside of the reservoir 24 to a delivery position located inside of the reservoir 24. FIG. 1 shows the mechanism 40 in the delivery position.

In an exemplary embodiment, the mechanism 40 includes a weight, also referred to as a weighted clunk, attached to the conduit 38. The weight can be attached to the conduit 38 by being integrally formed with the conduit 38. For example, the conduit 38 can have a thickened sidewall at a free (or distal) end 48 thereof so as to be heavier at the free end 48 than along a remainder of the conduit 38. Alternative to being integral with the conduit 38, the mechanism 40 can be a separate member fixedly attached to the conduit 38. For example, the mechanism 40 can be a metallic element (e.g., a ball, a ring, etc.) fixedly attached to the conduit 38 formed of a polymer that is lighter than the metal of the element. The metal can be, e.g., stainless steel or titanium. The mechanism 40 can be fixedly attached to the conduit 38 by being adhered thereto with adhesive, by being embedded within material forming the conduit 38, or by being attached to the conduit 38 using another attachment mechanism. In this exemplary embodiment in which the mechanism 40 includes a weight, the conduit 38 is formed of a flexible material that allows the conduit 38 to flex or bend within the reservoir 24. The weighted conduit 38 is therefore configured to flex or bend within the reservoir 24 as the pump's orientation changes, with the mechanism (weight) 40 facilitating the flexing or bending of the conduit 38 by the mechanism 40 being urged in a downward direction due to gravity (where “downward” indicates a direction toward the ground). Since the liquid drug 22 in the reservoir 24 naturally settles at a location in the reservoir 24 due to gravity, the weighted conduit 38 is configured to maintain complete communication with the liquid drug 22 regardless of the patient's orientation. In other words, the weighted conduit 38 is configured to “follow” the liquid drug 22 in the reservoir 24 to the liquid drug's settled location regardless of the patient's orientation.

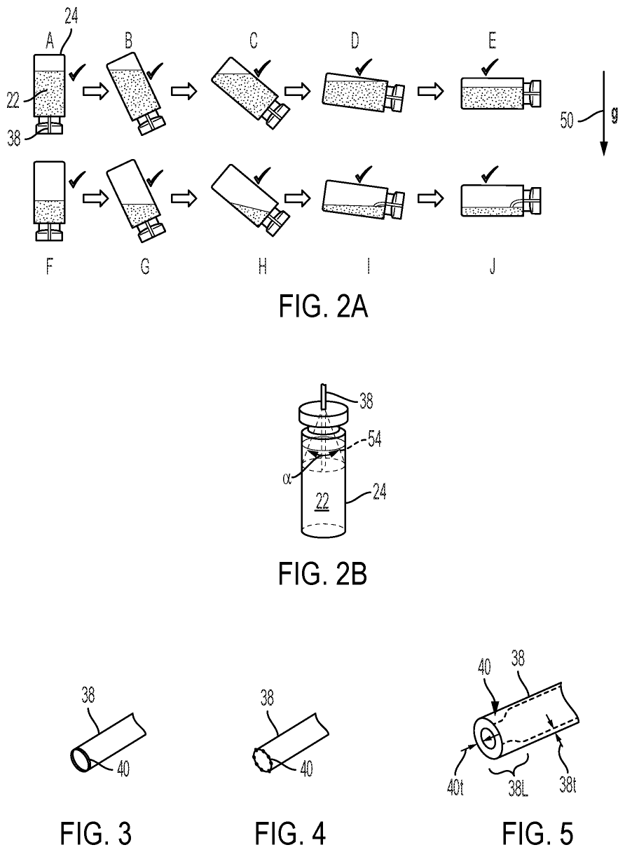

FIG. 2A illustrates ten possible relative positions A-J of the drug 22 and the conduit 38 in the reservoir 24. The reservoir 24 in an exemplary embodiment and as shown in FIG. 2A is a vial, but the reservoir 24 can have other forms, as will be appreciated by a person skilled in the art, such as a cartridge. A direction of gravity g is shown by arrow 50. Position A corresponds to the position of the drug 22 and the conduit 38 in the reservoir 24 with the pump 20 attached to the patient in accordance with the pump's provided instructions and with the patient standing or sitting upright, e.g., with the patient vertical. Position A is shown in FIG. 1. Position J corresponds to the position of the drug 22 and the conduit 38 in the reservoir 24 with the pump 20 attached to the patient in accordance with the pump's provided instructions and with the patient lying down, e.g., with the patient horizontal. Positions A-J are sequential positions as the patient moves from standing or sitting upright to lying down. Additional relative positions of the drug 22 and the conduit 38 in the reservoir 24 are possible between each of the illustrated ten positions A-J, and relative positions are also possible about other axes than the one illustrated in FIG. 2A, but are not shown for ease of illustration and discussion. In each of positions A-J, the conduit 38 is in complete communication with the drug 22, as indicated by the check mark next to each of positions A-J. In each of positions B-J the mechanism 40 is causing the conduit 40 to flex or bend to some degree in the reservoir 24 and thereby “follow” the settling of the drug 22 in the reservoir 24 as caused by gravity g. The flexing or bending of the conduit 38 is obscured by the drug 22 in positions B-H but is visible in positions I and J with the conduit 38 shown bending downward in the direction of gravity g.

FIG. 2B illustrates an area 54 of accessibility for the conduit 38 (with the mechanism 40 attached thereto) being in complete communication with the drug 22 in the reservoir 24. The reservoir 24 in an exemplary embodiment and as shown in FIG. 2B is a vial, but the reservoir 24 can have other forms, as will be appreciated by a person skilled in the art, such as a cartridge. The area 54 has a cone shape, in particular a right circular cone shape, with the conduit 38 extending along a central axis of the cone along a height of the cone. An angle a of the cone's apex to a point along the cone's circular base perimeter is about 30°. With the reservoir 24 oriented anywhere within the area 54 of accessibility, the conduit 38 can access about 99% of the drug 22 contained in the reservoir 24. A person skilled in the art will appreciate that a value may not be precisely equal to a value but nevertheless be considered to be about that value due to any number of factors, such as manufacturing tolerance and sensitivity of measurement equipment.

FIG. 3 illustrates an embodiment of the mechanism 40 as a weight attached to the conduit 38. The weight 40 in this illustrated embodiment includes a single ring at the free end 48 of the conduit 38 that extends fully around a circumference of the conduit 38. The weight 40 extending fully around the conduit's circumference may help ensure that the weight 40 follows gravity g regardless of the orientation of the pump 20 without kinking the conduit 38.

FIG. 4 illustrates another embodiment of the mechanism 40 as a weight attached to the conduit 38. The weight 40 in this illustrated embodiment includes a plurality of metallic elements arranged equidistantly around a circumference of the conduit 38 at the free end 48 thereof. The plurality of metallic elements being arranged equidistantly around a circumference of the conduit 38 may help ensure that the weight 40 follows gravity g regardless of the orientation of the pump 20 without kinking the conduit 38. The metallic elements are each a ball in this illustrated embodiment, but the metallic elements can have another configuration, e.g., a cube, an irregularly shaped element, an arc or C-shaped element that matches the curvature of the conduit's circumference, etc. The mechanism 40 includes eight metallic elements in this illustrated embodiment, but another number of metallic elements can be used.

FIG. 5 illustrates another embodiment of the mechanism 40 as a weight. The weight 40 in this illustrated embodiment includes a thickened sidewall of the conduit 38. The thickened sidewall extends along a partial longitudinal length 38L of the conduit 38 at the free (distal) end 48 of the conduit 38. In general, a thickness 40t of the thickened sidewall is greater than a thickness 38t of a remainder of the conduit's sidewall in order to provide weight at the free end 48 of the conduit 38. The thickness 40t of the thickened sidewall can be different for conduits with different diameters.

The mechanism 40 as a weight can have forms other than those illustrated in FIGS. 3-5. For example, the mechanism 40 as a weight can include a bulb attached to the free end 48 of the conduit 38.

Referring again to FIG. 1, in another exemplary embodiment, the mechanism 40 includes a fork at the free (or distal) end 48 of the conduit 38 that is located in the reservoir 24. In this exemplary embodiment, the conduit 38 includes a primary (or proximal) passageway in which the liquid drug 22 flows from the conduit 38 to the inlet flow path 30. The fork 40 includes a plurality of prongs that each includes a secondary (or distal) passageway in fluid communication with the primary passageway. Each of the secondary passageways includes a distal opening into which the liquid drug 22 enters before entering the primary passageway for delivery to the inlet flow path 30. The liquid drug 22 in the reservoir 24 can thus enter the primary passageway through any of the secondary passageways. The forked conduit 38 thus helps ensure that at least one of the prongs is in complete communication with the liquid drug 22 regardless of the patient's orientation and regardless of where the liquid drug 22 has settled within the reservoir 24 under the force of gravity.

In some circumstances in which at least one of the prongs is in complete communication with the liquid drug 22 within the reservoir 24, at least one other of the prongs may be in communication with air within the reservoir 24, particularly when a volume of the drug 22 in the reservoir 24 becomes low. Air may therefore enter the at least one of the prongs that is in communication with air within the reservoir 24. However, the fork 40 may increase the percentage of the drug 22 that the conduit 38 can access in the reservoir 24, thereby reducing chances of any of the prongs being in communication with air within the reservoir 24. In other words, the area 54 of accessibility of the conduit 38 (see FIG. 2B) may be increased due to the prongs' accessibility to the drug 22 within the reservoir 24 and/or the fork 40 may allow the conduit to access about 100% of the drug 22 contained in the reservoir 24.

Each of the prongs of the forked conduit 38 can include a weight similar to that discussed above. In such embodiments, the prongs can be configured to flex or bend within the reservoir 24 and to “follow” the liquid drug 22 in the reservoir 24 to the liquid drug's settled location regardless of the patient's orientation. The prongs being weighted may prevent any of the prongs from being in communication with air within the reservoir 24.

The pump 20 can include a sensor configured to detect air bubbles at one or more locations along the flow path of the drug 22 downstream of the reservoir 24. The sensor can be in operative communication with the control circuitry 36. In response to the control circuitry 36 receiving data from the sensor indicative of air bubbles being detected in the drug's flow path, the control circuitry 36 can trigger an error condition and thereby prevent any further delivery of the drug 22 to avoid air being introduced into the patient via the injector assembly 46. The error condition may require replacement of the pump 20 with a new pump.

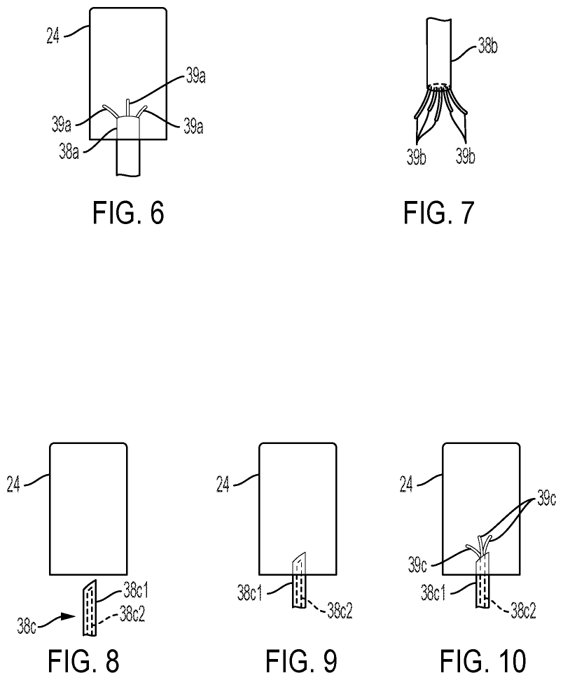

FIG. 6 illustrates an embodiment of the conduit 38 as a forked conduit 38a that includes the fork 40 with three prongs 39a. The forked conduit 38a in this illustrated embodiment is configured to be assembled with the prongs 39a located in the reservoir 24. FIG. 7 illustrates an embodiment of the conduit 38 as a forked conduit 38b that is configured and used similar to the conduit 38a of FIG. 6 except the fork 40 of FIG. 7 includes five prongs 39b instead of three prongs.

FIGS. 8-10 illustrate another embodiment of the conduit 38 as a forked conduit 38c that is configured to move from an initial position (shown in FIG. 8) located outside of the reservoir 24 to a delivery position (shown in FIG. 10) located inside of the reservoir 24. The forked conduit 38c in this illustrated embodiment includes the fork 40 with three prongs 39c but can include a different plural number of prongs 39c. The forked conduit 38c in this illustrated embodiment includes an outer member 38c1 and an inner member 38c2 slidably disposed in the outer member 38c1 and including the prongs 39c at a free (or distal) end thereof. In the initial position, the prongs 39c are fully contained within the outer member 38c1. The conduit 38c is configured to move from the initial position to an intermediate position, shown in FIG. 9, in which the conduit 38c has moved into the reservoir 24, e.g., by piercing through a septum at an end of the reservoir 24 under force provided by the pumping assembly 26. The prongs 39c are still fully contained within the outer member 38c1 with the conduit 38c in the intermediate position. The conduit 38c is configured to move from the intermediate position to the delivery position by the inner member 38c2 moving distally relative to the outer member 38c1, e.g., under force provided by the pumping assembly 26, such that the prongs 39c are located outside of the outer member 38c1. Alternatively, the conduit 38c can be configured to move from the intermediate position to the delivery position by the outer member 38c1 moving proximally relative to the inner member 38c2, e.g., under force provided by the pumping assembly 26, such that the prongs 39c are located outside of the outer member 38c1. The outer member 38c1 is configured to hold in the prongs 39c in a constrained (or unexpanded) configuration with the conduit 38c in the initial and intermediate positions. The prongs 39c are configured to move automatically from the constrained position to an unconstrained (or expanded) configuration in response to the conduit 38c moving from the intermediate position to the delivery position. To facilitate the prongs' automatic movement, the prongs 39c can be made from Nitinol or other shape memory material and can be biased to the unconstrained configuration.

Referring again to FIG. 1, in another exemplary embodiment, the mechanism 40 includes a telescoping cannula attached to the conduit 38. The telescoping cannula 40 includes one or more tubular members each configured to freely slide in and out of the free (or distal) end 48 of the conduit 38 that is located within the reservoir 24. A free (or distal) end of an innermost one of the tubular members (or the sole tubular member in embodiments with only one tubular member) has an opening therein into which the liquid drug enters for delivery from the reservoir 24 to the inlet fluid path 30 and eventually to the needle or cannula of the injector assembly 46. The pump 20 can be assembled with the free end of the conduit 38 and the free end of each of the tubular members located within the reservoir 24, or the free end 48 of the conduit 38 and the free end of each of the tubular members can be movable after pump 20 assembly from an initial position located outside of the reservoir 24 to a delivery position located inside of the reservoir 24. In this exemplary embodiment, the conduit 38 and each of the one or more tubular members is formed of a rigid material, e.g., a metal such as stainless steel or titanium, which may facilitate smooth sliding of the one or more tubular members in and out of the conduit 38. The one or more tubular members are configured to freely slide in and out of the conduit 38 as the pump's orientation changes, with the movement of the one or more tubular members being caused by gravity. Since the liquid drug 22 in the reservoir 24 naturally settles at a location in the reservoir 22 due to gravity, the one or more tubular members are configured to maintain complete communication with the liquid drug 22 regardless of the patient's orientation.

The telescoping cannula 40 can include a weight similar to that discussed above. The weight is configured to encourage movement of the telescoping cannula in response to gravity. In such embodiments, the telescoping cannula 40 can be configured to flex or bend within the reservoir 24 and to “follow” the liquid drug 22 in the reservoir 24 to the liquid drug's settled location regardless of the patient's orientation. Each of the telescoping's cannula's tubular members can include a weight, or only a partial number of the telescoping's cannula's tubular members can include a weight. In an exemplary embodiment, at least an innermost one of the tubular members includes a weight.

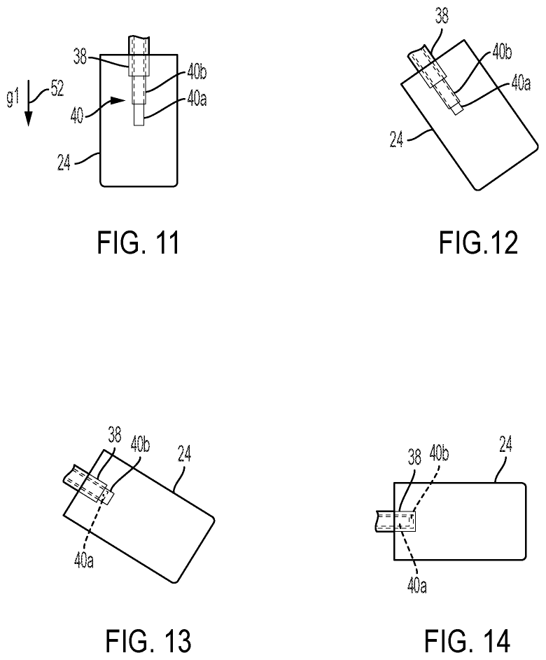

FIGS. 11-14 illustrate an embodiment of the mechanism 40 as a telescoping cannula including two tubular members 40a, 40b. The telescoping cannula 40 in this illustrated embodiment includes two tubular members 40a, 40b freely slidably disposed within the conduit 38, but as mentioned above, the telescoping cannula 40 can include another number of tubular members, e.g., one, three, etc. The inner one of the tubular members 40a is freely slidably disposed within the outer one of the tubular members 40b in telescoping fashion. A free (or distal) end of the inner one of the tubular members 40a has an opening therein into which the liquid drug enters for delivery from the reservoir 24 to the inlet fluid path 30 and eventually to the needle or cannula of the injector assembly 46.

FIGS. 11-14 illustrate four possible relative positions of the tubular members 40a, 40b and the conduit 38 in the reservoir 24. A direction of gravity g1 is shown by arrow 52 in FIG. 11, with gravity g1 also being downward in FIGS. 12-14. FIG. 11 corresponds to the position of the drug 22, the conduit 38, and the tubular members 40a, 40b in the reservoir 24 with the pump 20 attached to the patient in accordance with the pump's provided instructions and with the patient standing or sitting upright, e.g., with the patient vertical. The tubular members 40a, 40b in FIG. 11 are each at their fully extended positions in which the inner tubular member 40a is extended as far as possible out of the outer tubular member 40b and the conduit 38, and the outer tubular member 40b is extended as far as possible out of the conduit 38. FIG. 14 corresponds to the position of the drug 22, the conduit 38, and the tubular members 40a, 40b in the reservoir 24 with the pump 20 attached to the patient in accordance with the pump's provided instructions and with the patient lying down, e.g., with the patient horizontal. The tubular members 40a, 40b in FIG. 14 are each at their fully retracted positions in which the inner tubular member 40a is located as far as possible inside the outer tubular member 40b and the conduit 38, and the outer tubular member 40b is retracted as far as possible inside the conduit 38. Additional relative positions of the drug 22, the conduit 38, and the tubular members 40a, 40b in the reservoir 24 are possible between each of the illustrated four positions, and relative positions are also possible about other axes than the one illustrated in FIGS. 11-14, but are not shown for ease of illustration and discussion. In each of FIGS. 11-14, the conduit 38 is in complete communication with the drug 22 via the passageway of the inner tubular member 40a.

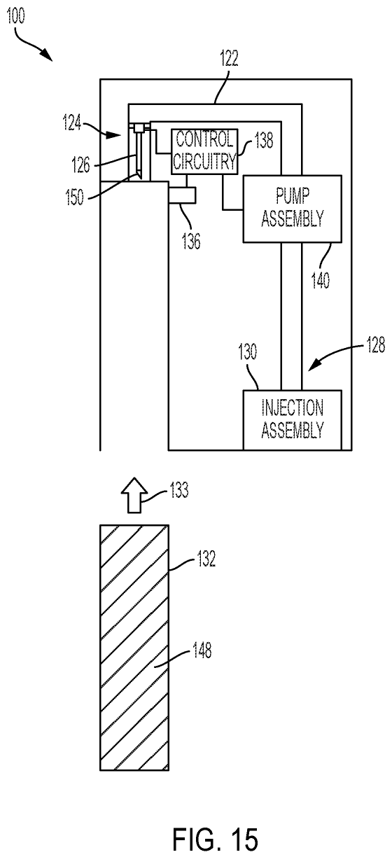

FIGS. 15-17 illustrate another embodiment of a pump 100, e.g., a patch pump, configured to be worn by a patient and to deliver a drug 148 to the patient. The pump 100 of FIGS. 15-17 is generally configured and used similar to the pump 20 of FIG. 1. The pump 100 is configured to engage with a prefilled therapeutic substance reservoir 132. Within the pump 100 is a sterile fluid path 122 for delivering a drug 148 from the reservoir 132 to a patient wearing the pump 100. The sterile fluid path 122 has a conduit 126 at an upstream end 124 of the sterile fluid path 122 and has an injection assembly (also referred to herein as an “injector assembly”) 130 at a downstream end 128 of the sterile fluid path 122. The conduit 126 includes a mechanism 150 configured to ensure that the conduit 126 is in complete communication with the drug 148 in the reservoir 132 at least when the conduit 126 is receiving the drug 148 therein from the reservoir 132, e.g., under force of an electromechanical pumping assembly 140 of the pump 100, regardless of an orientation of the patient wearing the pump 100, e.g., regardless of whether the patient is standing, sitting, lying down, bent over, etc.

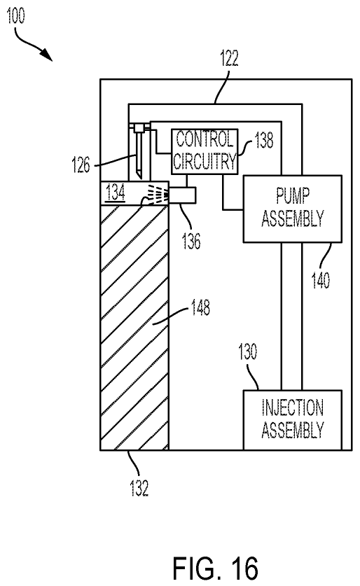

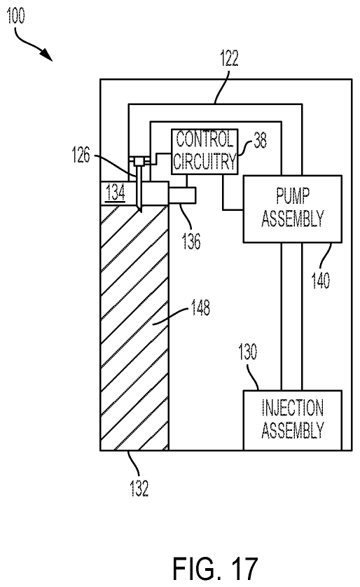

The pump 100 and the prefilled therapeutic substance reservoir 132 are configured to engage with one another, such as shown by arrow 133 in FIG. 15, e.g., the reservoir 132 is configured to be inserted into the pump 100. When the pump 100 and the reservoir 132 are engaged with one another, such as is shown in FIG. 16, a sealed disinfection chamber 134 is defined between the sterile fluid path 122 and the reservoir 132. While the pump 100 and the reservoir 132 are typically sterile, the disinfection chamber 134 is (a) initially non-sterile, and (b) typically sealed from further bacteria or virus penetration. The conduit 126, led by the mechanism 150, is configured to be driven to penetrate the disinfection chamber 134 and subsequently the reservoir 132 when the pump 100 and the reservoir 132 are engaged with one another, such that fluid communication is established between the reservoir 132 and the sterile fluid path 122, such as is shown in FIG. 17.

The pump 100 includes a disinfection assembly 136 configured to disinfect the disinfection chamber 134 prior to the conduit 126 penetrating the disinfection chamber 134 and thus before the conduit 126 enters the reservoir 132. The pump 100 includes control circuitry 138 configured to activate the disinfection assembly 136, to subsequently terminate the activation of the disinfection assembly 136, and to then drive the conduit 126, led by the mechanism 150, to penetrate the disinfection chamber 134 and subsequently the reservoir 132.

Once fluid communication is established between the reservoir 132 and the sterile fluid path 122, the control circuitry 138 is configured to drives a pump assembly 140 to draw the drug 148 from the reservoir 132 and deliver it to the patient via injection assembly 130 similar to that discussed above regarding the control circuitry 36 and the injector assembly 46 of FIG. 1.

The mechanism 150 can have a variety of configurations, as discussed above, such as a weight or a fork. In an exemplary embodiment, the mechanism 150 includes a weight attached to the conduit 126. The mechanism 150 being a weight is configured to help the conduit 126 enter the reservoir 132 by being sturdier than a remainder of the conduit 126. For example, the mechanism 150 can be a pointed metal distal tip that leads a flexible proximal portion of the conduit 126 into through a penetrable septum and into the reservoir 132.

The pumps described herein can include a user interface configured to provide for interaction with a user. The user interface can be implemented on a computer having a display screen, such as for example a cathode ray tube (CRT) or a liquid crystal display (LCD) or a light emitting diode (LED) monitor for displaying information to a user. The display screen can allow input thereto directly (e.g., as a touch screen) or indirectly (e.g., via an input device such as a keypad or voice recognition hardware and software). The user interface can take the form of, e.g., a touchscreen or a keypad.

As discussed herein, one or more aspects or features of the subject matter described herein, for example components of the control circuity or user interface, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein, for example a user interface of a pump as described herein, can be implemented on a computer having a display screen, such as for example a cathode ray tube (CRT) or a liquid crystal display (LCD) or a light emitting diode (LED) monitor for displaying information to a user. The display screen can allow input thereto directly (e.g., as a touch screen) or indirectly (e.g., via an input device such as a keypad or voice recognition hardware and software).

The present disclosure has been described above by way of example only within the context of the overall disclosure provided herein. It will be appreciated that modifications within the spirit and scope of the claims may be made without departing from the overall scope of the present disclosure.

1. A pump configured to deliver a drug to a patient, comprising:
a reservoir configured to contain a liquid drug therein;
a conduit configured to receive the drug therein from the reservoir, the conduit having a weight at a free end thereof;
a needle configured to be inserted into a patient; and
a pumping assembly configured to drive the liquid drug into the conduit from the reservoir and into the needle for delivery of the liquid drug into the patient.

2. The pump of claim 1, wherein the conduit is a flexible tube.

3. The pump of claim 2, wherein the flexible tube is defined by a circumferential sidewall extending along a length of the flexible tube, and the weight is a thickened portion of the sidewall at the free end of the flexible tube.

4. The pump of claim 3, wherein the flexible tube is formed of a polymer.

5. The pump of claim 2, wherein the weight is a metallic element attached to the free end of the flexible tube.

6. The pump of claim 5, wherein the flexible tube is formed of a polymer.

7. The pump of claim 1, wherein the free end of the conduit is freely movable in the reservoir in response to gravitational force.

8. The pump of claim 1, further comprising control circuitry configured to cause activation of the pumping assembly and thereby move the liquid drug into the conduit from the reservoir and from the conduit into the needle.

9. The pump of claim 1, wherein the pump is assembled with the weight located within the reservoir.

10. The pump of claim 1, wherein the pump is assembled with the weight located outside of the reservoir.

11. The pump of claim 10, further comprising control circuitry configured to drive the weight from outside of the reservoir to inside of the reservoir.

12. The pump of claim 1, wherein the pump is configured to be worn by a patient.

13. The pump of claim 1, wherein the liquid drug is one of an antibody, a hormone, an antitoxin, a substance for control of pain, a substance for control of thrombosis, a substance for control of infection, a peptide, a protein, human insulin or a human insulin analogue or derivative, polysaccharide, DNA, RNA, an enzyme, an oligonucleotide, an antiallergic, an antihistamine, an anti-inflammatory, a corticosteroid, a disease modifying antirheumatic drug, erythropoietin, and a vaccine.

14. A method of using the pump of claim 1, comprising:
activating the pumping assembly to move the liquid drug into the conduit from the reservoir and from the conduit into the needle.

15. The method of claim 14, wherein the pump further comprises control circuitry configured to cause the activation of the pumping assembly.

16. The method of claim 14, wherein the liquid drug is one of an antibody, a hormone, an antitoxin, a substance for control of pain, a substance for control of thrombosis, a substance for control of infection, a peptide, a protein, human insulin or a human insulin analogue or derivative, polysaccharide, DNA, RNA, an enzyme, an oligonucleotide, an antiallergic, an antihistamine, an anti-inflammatory, a corticosteroid, a disease modifying antirheumatic drug, erythropoietin, and a vaccine.

17. A pump configured to deliver a drug to a patient, comprising:
a reservoir configured to contain a liquid drug therein;
a conduit configured to receive the drug therein from the reservoir, the conduit includes a single proximal passageway therein, a free end of the conduit includes a plurality of tubular prongs each distal to the single proximal passageway and each including a secondary passageway therein, each of the secondary passageways being in fluid communication with the single proximal passageway;
a needle configured to be inserted into a patient; and
a pumping assembly configured to drive the liquid drug into the conduit from the reservoir and into the needle for delivery of the liquid drug into the patient.

18. (canceled)

19. (canceled)

20. The pump of claim 17, further comprising control circuitry configured to cause activation of the pumping assembly and thereby move the liquid drug into the conduit from the reservoir and from the conduit into the needle.

21. (canceled)

22. (canceled)

23. (canceled)

24. (canceled)

25. (canceled)

26. A method of using the pump of claim 17, comprising:
activating the pumping assembly to move the liquid drug into the conduit from the reservoir and from the conduit into the needle.

27. (canceled)

28. (canceled)

29. A pump configured to deliver a drug to a patient, comprising:
a reservoir configured to contain a liquid drug therein;
a conduit configured to receive the drug therein from the reservoir;
a telescoping cannula configured to slide in and out of the conduit in response to gravitational force;
a needle configured to be inserted into a patient; and
a pumping assembly configured to drive the liquid drug into the conduit from the reservoir and into the needle for delivery of the liquid drug into the patient.

30. (canceled)

31. (canceled)

32. The pump of claim 29, further comprising control circuitry configured to cause activation of the pumping assembly and thereby move the liquid drug into the telescoping cannula from the reservoir, from the telescoping cannula into the conduit, and from the conduit into the needle.

33. (canceled)

34. (canceled)

35. (canceled)

36. (canceled)

37. (canceled)

38. A method of using the pump of claim 29, comprising:
activating the pumping assembly to move the liquid drug into the telescoping cannula from the reservoir, from the telescoping cannula into the conduit, and from the conduit into the needle.

39. (canceled)

40. (canceled)
